(19) United States
(12) Patent Application Publication
(10) Pub. No.: US 2011/0098523 A1
(43) Pub. Date: Apr. 28, 2011

(54) Apparatus for Stimulating a Healing Process in the Region of an Implant

(75) Inventors: Werner Kraus, Muenchen (DE); Stephanie Kraus-Geiges, Muenchen (DE); Heribert Stephan, Muenchen (DE)

(73) Assignee: Neue Magnetodyn GmbH

(21) Appl. No.: 12/990,244

(22) PCT Filed: Mar. 19, 2009
(86) PCT No.: PCT/EP09/02048

§ 371 Date: Jan. 5, 2011

(30) Foreign Application Priority Data
Apr. 30, 2008 (DE) 10 2008 021 574.0

(51) Int. Cl. A61N 2/02

(52) U.S. Cl. 600/13

(57) ABSTRACT

An apparatus is provided for stimulating a healing process. The apparatus includes a coil arrangement which is coupled to a functional power generator in order to generate an electromagnetic field in an affected body region, a control unit for influencing a voltage curve generated by the functional power generator in accordance with signals transmitted by an input interface of the control unit, at least one implant that is disposed in the affected body zone and is coupled to a transformer coil arrangement, the poles of which are connected to a couple of electrodes, a sensing device for sensing a voltage applied between the couple of electrodes, and a transmission device for transmitting signals characteristic of the voltage to the control unit.

BACKGROUND AND SUMMARY OF THE INVENTION

The invention relates to an apparatus for stimulating a healing process in the region of an implant.

According to the technology pursuant to Kraus and Lechner, it is known to integrate coil arrangements referred to as pick-up or transformer coils into the components of support metal osteosynthesis devices or joint endoprostheses and to electrically connect their poles to implant sections serving as electrodes. In this arrangement, a voltage effectively supporting the healing process is induced in the meson field of an external magnetic coil in connection with a functional power generator.

One example of an osteosynthesis device making use of the described technology is disclosed in DE 10 2006 018 191 A1. The femoral head cap implants described in DE 10 2004 024 473 A1 are examples for the use of the technology in joint endoprosthetics.

When putting the described technology into practice, the below aspects must, in particular, be taken into account.

A first aspect relates to the spatial allocation of the axes of the induced magnetic coil field to the axis or plane of the pick-up coil effectively pervaded by the inducing magnetic field. When using electro osteosynthesis and electro joint endoprostheses, the pick-up coil becomes a mechanically fixed component of the implant by being cemented or inserted into, for example, the drilled hole of cannulated bone screws or by being screwed into the end cap of a bone marrow nail containing the pick-up coil. The anchoring of the implant in the bone, however, is primarily determined by the requirements of an optimum support function of its fragments. This results in a variation range of the solid angle of the winding axis of the pick-up coil in the bone support implant relative to the longitudinal bone or body axis of 0° to 90°. In comparison, the directional angle of the inducing magnetic field in an external magnetic coil is relatively restricted: for a solenoid form, in which, in particular, extremities are located within a solenoid coil, the vector of the inducing coil core field extends parallel to the longitudinal bone axis. In the case of a Helmholtz arrangement in contrast, if body parts are located in the space between of a pair of Helmholtz coils, the vector of the coil core field extends perpendicular to the longitudinal bone axis. In the case of the pick-up coil in an osteosynthesis screw, for example, for fixing an oblique fracture, an angle of 45° between the magnetic field of the inducing solenoid or Helmholtz coil and the pick-up coil of the implant is obtained in the least advantageous case. This results in an approximately 30% loss of the induced electric voltage. Therefore, if the system is configured to the target value of 700 mV required for the bone stimulation under optimum geometric conditions, only scarcely 500 mV will be obtained in the described suboptimal but realistic situation. Comparable conditions also prevail in connection with endoprosthetics.

A second aspect relates to the frequency of the waveforms of the inducing magnetic field, as this constitutes another important biological efficacy factor. It has been shown that the cellular messengers Ca²⁺ and cAMP relevant for activating connective tissue and bone cells follow a resonance of vibrations of their density at 15 Hz. Therefore, the frequency range of 1 to 30 Hz, but most preferably 10 to 20 Hz of the electromagnetic induction should be maintained so that the frequency of the vibrations can hardly act as a parameter for influencing the therapeutic situation.

A third aspect relates to the signal form of the inducing magnetic field which determines the cellular efficacy factor of the chronological sequence of the magnetic flux density in the affected body region. The optimal stimulating effect on the competent cells (fibroblasts and osteoblasts) regarding their differentiation, their metabolism, and their synthesis of structural proteins was obtained by sinoidal progressions. They showed a considerable superiority with respect to their effectivity value and the impedance as compared to the effectiveness of other systems in which induction is obtained by spike pulse bursts or square waves with higher order harmonics of more than 100 kHz and a distinctive unsteadiness (discontinuities) of their chronological progressions.

The object of the invention is to further develop the technology of the magnetically induced electro-osteostimulation in an enhanced manner so that the induced voltage reaches the physiologically desired target voltage even under suboptimal geometric conditions, and further, an increase of the desired magnetic flux density is excluded, the resonance frequency of cellular messengers (Ca²⁺ and cAMP) of 15 Hz is maintained within a clinically proven frequency spectrum of 2 to 30 Hz for the exciting vibration, and the vibration or signal form of the inducing electromagnetic field runs continuously and free of unsteadiness (discontinuities).

The invention consists of an apparatus for stimulating a healing process comprising a coil arrangement coupled to a functional power generator for generating an electromagnetic field in an affected body region, a control unit for influencing a voltage curve generated by the functional power generator dependent on signals transmitted to an input interface of the control unit, at least one implant located in the affected body region and coupled to a transformer coil arrangement, the poles of the transformer coil arrangement being connected to a pair of electrodes, a sensing device for detecting an electric voltage existing between the electrodes of the electrode pair, a transmission device for transmitting signals characteristic to the electric voltage to the control unit. Thus, it is possible to respond to an induction voltage falling below the desired voltage value and to ultimately set the desired voltage by influencing the voltage curve generated by the waveform generator. Sometimes this can be achieved by a minor change of the voltage curve while otherwise maintaining the characteristics of the magnetic field so that no increase of the flux density and no change of the fundamental frequency are required. The apparatus according to the invention is, inter alia, characterised by a sensing device and a transmission device. In special cases, said devices can be integrated with each other or identical.

Preferably, the transmission device comprises at least one transmitter for wireless communication with a receiver associated with the input interface of the control unit. Such a transmitter may have various designs. It is important that it translates the measured values detected by the sensing device so that corresponding signals can be transmitted to the receiver of the control unit. One option for the transfer of information from the transmitter to the receiver in the control unit is the active generation of transmission signals dependent on the measured values of the sensor arrangement.

It may, however, also be contemplated that the transmission device comprises at least one RFID transponder the information content of which is detectable by a reading device associated with the input interface of the control unit. An RFID transponder is a device which can only “transmit” information through interaction with a reading device. For this purpose, the RFID transponder ultimately receives an electromagnetic high frequency field generated by the reading device in order to then change it depending upon information stored in the RFID transponder. The change is then detected by the reading device. Due to this very limited functionality of an RFID transponder, it is inexpensive and space-saving in comparison to conventional active transmitters.

The information transfer from the RFID transponder to the reading device can take place, as the readable information content of the RFID transponder being changeable dependent on signals supplied by the sensor arrangement. In the simplest case, the sensing device applies different voltages to the memory of the RFID transponder, wherein said voltages depending on the voltage detected by the sensing device, or is the detected voltage itself; in the latter case, the sensing device and the transmission device are to be referred to as integrated or identical. Different voltages can now cause the content of the memory of the RFID transponder to change so that ultimately the identification transmitted to the reading device by the RFID transponder is also changed. The use of writable RFID transponders is required to enable a change of the content of the memory of the RFID transponder.

Alternatively or additionally, however, it is also possible to provide a plurality of RFID transponders which can be activated or deactivated dependent on signals supplied by the sensor device. In this case, non-writable transponders are sufficient. One or more threshold circuits integrated in the sensing device and the RFID transponders make sure that different RFID transponders are active or inactive dependent on the supplied voltage. Thus, the reading device can also receive different identifications dependent on the voltage and therefore, ensure that the functional power generator generates a voltage curve adjusted to the detected characteristics of the affected body region.

The invention is further enhanced in a particularly advantageous manner in that the coil arrangement comprises a coil comprising a coil winding having an intersection point which defines two surfaces by a figure eight shaped form, the surfaces being aligned relative to each other so that the magnetic fields generated by a current flow in the coil arrangement and pervading the surfaces are substantially rectified. The effect of two separate induction coils having the same direction of their magnetic field in which the injured body region is located can be obtained with a single coil in this manner. This renders the application comfortable, particularly owing to the reduced mechanistic complexity.

It is particularly useful that the coil arrangement is flexible so that the surfaces can be positioned on the opposing sides of the affected body region. Owing to its flexible winding which may, in particular, result from the elasticity of the used material, reshaping of the coils to obtain a figure eight or infinity symbol becomes possible. The “loops” of the coil resulting from said reshaping of the coil winding may, for example, be positioned at both sides of an extremity. Depending on the application, they may have the same or different sizes. The coil arrangement can also be used very versatilely thanks to its flexibility. In connection with the design of the coil arrangement, it may be contemplated that fasteners for establishing and maintaining the alignment of the surfaces in respect to each other are provided in two positions of the coil arrangement facing away from the intersection point. The fasteners may, for example, be belts, snaps, hook-and-loop fasteners, buckles or the like. In a particularly advantageous manner, it may be contemplated that the intersection point of the coil arrangement is fixable by means of a coupling device. Such a coupling device may, for example, be realised by an elastic strap having a hook-and-loop fastener or a belt buckle. It is particularly advantageous that the position of the intersection point, and thus, the dimensions of the surfaces are variable. As the surface ratio of the coil loops can be adjusted in this manner, the magnetic induction flux density, which is defined as the quotient of the magnetic flux and the observed surface, is also adjustable. The ratio of the induction flux densities is the reciprocal value of the ratio of the respective surfaces. The surface ratio of the two loops may be selected according to the therapeutic requirements by changing the fixation of the intersection point by a variable positioning of the coupling device. For example, a high magnetic induction flux density may be obtained in a target area of the body by positioning a small-surface coil loop in its proximity, while with regards to the other, large-surface coil surface, its parallel arrangement relative to the smaller surface must primarily be regarded in order to provide the Helmholtz coil effect. With such a coil arrangement, it is accomplished that the magnetic field of the two loops of the coil arrangement opposite each other is rectified by the spatial reversal of the current direction in one of the two loops similar to the arrangement of two separate coils according to Helmholtz, whereby a particularly good and flexible manageability is achieved. Convient handling during the adjustment of the geometric shape of the coil arrangement to the position of the respective bones or soft tissue lesion is possible. For example, the loop surfaces can be adjusted to treatment areas such as foot, knee, lower leg, thigh, pelvis, spine, hand, lower arm, upper arm, jaw and skull regions by varying the loop shapes and sizes. The loop shapes and sizes can also be varied in respect to the intensity of the magnetic field. Another feature of the coil arrangement according to the invention is an increase of the flux density in the area proximate to the intersection point so that a relatively strong magnetic field can be concentrated on a small body surface. Thus, highly localised diseases such as abscesses and infections can be treated effectively.

In a particularly advantageous manner, the invention is further developed in that the functional power generator is capable of generating virtually purely harmonic voltage curves having a first harmonic wave component or abnormal harmonic voltage curves having a second harmonic wave component which is larger than the first harmonic wave component dependent upon the signals transmitted to the input interface of the control unit. By changing the voltage curve generated by the functional power generator, the proportion of harmonic waves in the electromagnetic field in the affected body region can be varied. In particular, the frequency of the low-frequency magnetic field can be maintained in this case whereas electric fields depending on the temporal differential of the magnetic field can be varied up to high frequencies. In this way, the affected body region can be exposed to virtually invariable low-frequency alternating magnetic fields promoting the differentiation of the cells, while high-frequency components are generated when appropriate because of reduced induction voltages.

In concrete terms for the stimulation in the region of implants, it is contemplated that the functional power generator is capable of generating virtually purely harmonic voltage curves in the presence of a voltage detected by the sensing device which corresponds to a target voltage or is within a target voltage interval and to generate abnormally harmonic voltage curves in case of a dropping below the target voltage or the target voltage interval.

In this connection, it is particularly useful that the functional power generator is integrated in a control that regulates the voltage detected by the sensing device to a target voltage or a target voltage interval by changing the voltage curves between purely harmonic and abnormally harmonic.

A particularly preferred embodiment of the apparatus consists in that a plurality of implants, corresponding transformer coil arrangements, corresponding pairs of electrodes, corresponding sensing devices, and corresponding transmission devices are provided, that the functional power generator can be influenced on the basis of the smallest voltage measured by the sensing device or the smallest voltage measured by the sensing device which is still above a minimum voltage value and in that voltage limiters are allocated to the pairs of electrodes. If it is desired to generate the physiologically particularly effective voltage of, for example, 700 mV in each of the implants, in which the transformers sometimes have differing angles to the direction of the magnetic field, it is required to influence the wave form of the exciting magnetic fields bytaking the transformer coil located in the least advantageous position in respect to the magnetic field into account. Without appropriate countermeasures, this would result in the voltage values being excessive at other transformers better positioned in respect to the magnetic field. Therefore, this is prevented by voltage limiters associated with the individual transformers. If particular transformers exhibit a very low voltage, it could, however, also be reasonable not to take them into account. Only those transformer voltages which exceed a specific threshold value of, for example, 200 mV or 300 mV would then be taken into account as the basis for influencing the waveform.

The present invention relates to an apparatus for stimulating a healing process. The invention could, however, also be formulated as a method in which the following process steps are of significant relevance: generating an electromagnetic field in the area of an implant coupled with a transformer coil arrangement, the poles of the transformer coil arrangement being connected to a pair of electrodes; detecting a voltage generated by the transformer coil arrangement; transmitting signals characteristic to the electric voltage to a control unit of a functional power generator; influencing a voltage curve generated by the functional power generator dependent on the transmitted signals. Particularly advantageous embodiments of this method are characterised in that the transfer of the information to the control unit is effected using at least one RFID transponder. Furthermore, the method is particularly advantageous if the functional power generator is integrated in a control unit regulating the detected voltage to a target voltage or into a target voltage interval by changing the voltage curves between purely harmonic and abnormally harmonic.

### BRIEF DESCRIPTION OF THE DRAWINGS

The invention will now be discussed by way of example with the aid of particularly preferred embodiments with reference to the accompanying drawings in which:

FIG. 1 shows an apparatus according to the invention during its application;

FIG. 2 shows an implant associated with the apparatus according to the invention;

FIG. 3 shows a purely harmonic voltage curve;

FIG. 4 shows an abnormal harmonic voltage curve;

FIG. 5 shows a coil arrangement suitable for an apparatus according to the invention in a first state;

FIG. 6 shows a coil arrangement suitable for an apparatus according to the invention in a second state;

FIG. 7 shows a schematic representation for explaining the spatial alignment of a magnetic field generated by a coil arrangement according to the invention;

FIG. 8 shows a cut perspective partial representation of a coil arrangement having plastic properties; and

FIG. 9 shows an arrangement comprising a plurality of implant components for explaining another aspect of the invention.

### DETAILED DESCRIPTION OF THE DRAWINGS

In the following description of the drawings, identical reference numerals designate the same or comparable components.

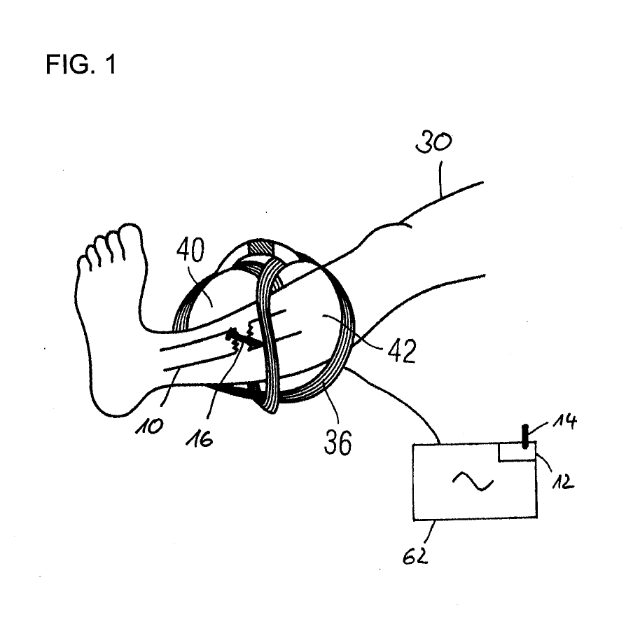

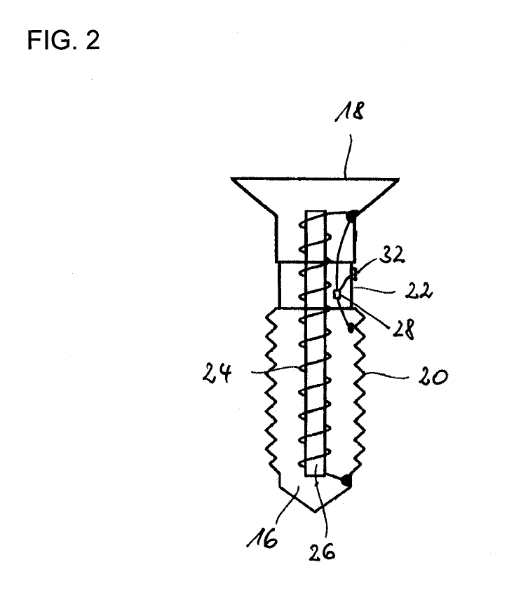

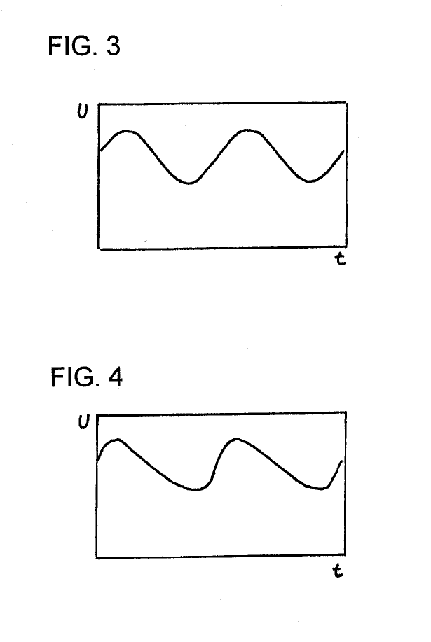

FIG. 1 shows an apparatus according to the invention during its application. FIG. 2 shows an implant associated with the apparatus according to the invention. FIGS. 3 and 4 show signal waveforms which can be generated by the apparatus according to the invention. In FIG. 1, a leg 30 with a broken lower leg bone 10 can be seen. The induced electromagnetic field is generated by a functional power generator 62 in a flexible magnetic coil 36. For a better illustration, the flexible coil 36 is shown at a distance to the affected body region. In practice, the magnetic coil 36 can, owing to its flexibility, be directly formed to the body or a bandage so that its winding immediately surrounds the injured area.

The fragments of the lower leg bone are connected to each other and supported by an implant 16. The implant 16 may, in particular, be a bone screw in or on which the components required for stimulation are positioned. A cross sectional view of such a bone screw 16 is shown in FIG. 2. The bone screw 16 comprises an electrically conductive proximal area including a screw head 18, a distal area provided with a thread 20, and an insulating area 22 located between between the proximal and the distal area. The electrically conductive areas are separated by the insulating area 22 and are also connected to the poles of a transformer coil 24 which has a magnetically soft core 26. Further, a sensing device 28 is indirectly or directly electroconductively connected to the poles of the transformer coil 24. Likewise, an RFID transponder 32 is provided which is coupled to the sensing device 28 via a cable. Thus, the information stored in the RFID transponder 32 can be changed depending upon the voltage induced in the transformer coil 24 so that a measurement for the induced voltage can be detected via a reading device 14 of the control unit 12 associated with the functional power generator 62. In order to enable a unproblematic reading of RFID transponder 32, it is located outside of the bone screw 16 in the present embodiment, i.e. not in the bone screw 16. Thus, shielding of the high frequency fields required for reading the RFID transponder 32 is avoided. However, it is also feasible to position the RFID transponder 32 at an appropriate location within the screw, particularly inside of the insulating area 22. Therefore, it is possible with the present arrangement to successfully measure the induced electrical voltage U_{ind}=dB/dt directly or indirectly and to transmit it to a control unit in the functional power generator of the inducing magnetic coil which preferably is comprised of a micro processor. The control unit 12 is responsible for the control of the functional current of the inducing magnetic coil, and it responds to the RFID signal by changing the signal waveform (dB/dt) from the chronologically symmetric sinewave progression to a chronologically unsymmetric progression of its rising or falling flank of the current of the inducing magnetic coil. FIG. 3 shows the unmodulated fundamental waveform, while FIG. 4 shows a modulated waveform with a steepened rising flank. In comparison with the voltage induced by a chronologically symmetrical sinewave free of harmonic waves which is, for example, smaller than the target voltage by an amount of 200 mV, the steepness (dB/dt) of one flank of the sinewave is increased until the target value U_{ind}=700 mV is reached at the output of the pick-up coil. This can be achieved by a control. Each of the signal waveforms shown in FIGS. 3 and 4 fulfill the requirements to generate harmonics of the fundamental sine waveform of a maximum of 1 kHz, preferably to only 500 Hz at a maximum flux density of 3 mT and to have the resonance frequency of cellular messengers as the fundamental waveform. To guarantee a smooth transmission of the high frequency electromagnetic signals between the RFID transponder 18 and the input interface of the control unit 12 configured as the reading device 14, it may be required to interrupt the generation of electromagnetic fields in the magnetic coil 36 for a short period of time for the purpose of communication. The particular advantage of the use of RFID transponders in comparision to a conventional transmitter capable of actively transmitting signals dependent on the generated voltage becomes apparent here. Namely, the RFID transponder(s) work on the basis of stored information so that it is not required to maintain the exciting magnetic field to obtain information relating to the induced voltage.

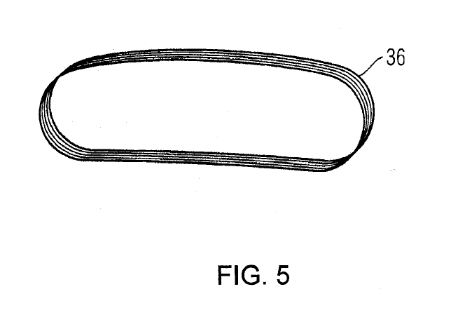

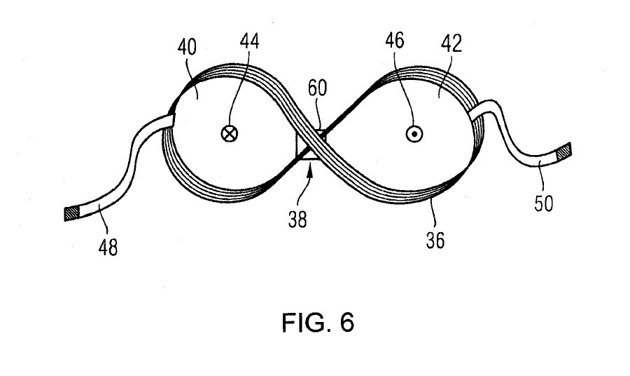

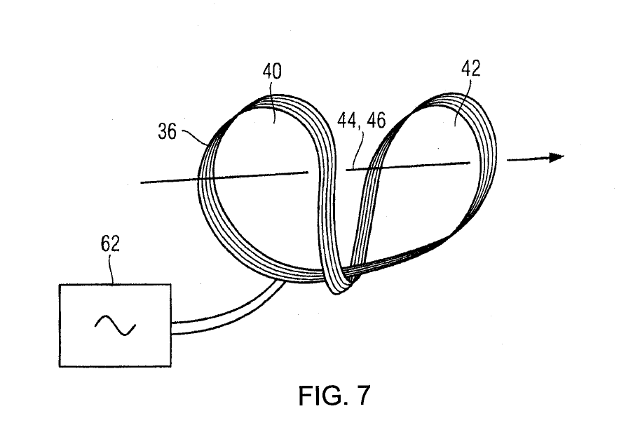

FIG. 5 shows a coil arrangement suitable for an apparatus according to the invention in a first state. FIG. 6 shows a coil arrangement suitable for an apparatus according to the invention in a second state. FIG. 7 shows a schematic representation for explaining the spatial alignment of a magnetic field generated by a coil arrangement according to the invention. If the flexibility of the coil arrangement 36 is used to arrange it in a figure eight, two surfaces 40, 42 as well as an intersection point 38 are defined. The intersection point 38 is fixed by a coupling device 60 and can preferably be shifted so that the ratio of the surfaces 40, 42 is variable. For example, an elastic belt provided with a loop-and-hook fastener or a belt buckle can serve as the coupling device 60. It is also feasible to equip the surfaces of the coil arrangement so that they form the components of a loop-and-hook fastener and can, in this way, directly adhere to each other in different positions. To facilitate adjustment, marks or scales can be provided in the variation range. The user can then refer to the marks if he wishes to implement a specific fixation of the intersection point 38. If an alternating current flows through such a coil arrangement 36, the induced magnetic fields will have opposite directions as indicated by the vector symbols 44, 46. Owing to the flexibility of the coil arrangement 36, it can, however, also be arranged in another shape. This is shown in connection with FIG. 1 in which a leg having a broken lower leg bone 10 is shown as an example for an affected body region. The surfaces 40, 42 then face each other, and the magnetic fields generated in the respective segments of the coil have the same direction. This is illustrated again in FIG. 7, in which a functional power generator 62 coupled to the coil arrangement is also shown. Namely, the uniform magnetic field vector 44, 46 pervades both surfaces 40, 42. According to FIG. 1, the magnetic field pervades the affected body region primarily perpendicular to the longitudinal axis of the extremity. It is also possible that the extremity runs through the surfaces defined by the loops of the figure eight shaped coil so that the magnetic field is then primarily parallel to the axis of the extremity.

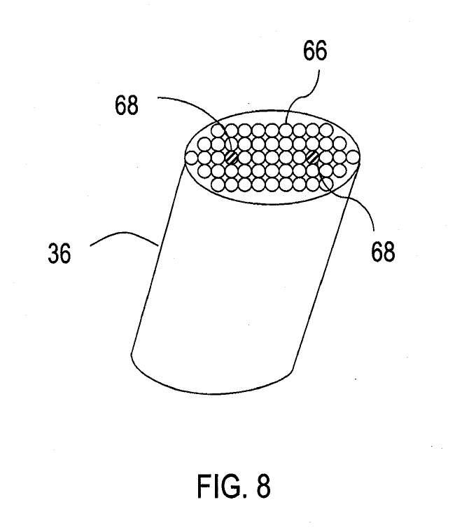

FIG. 8 shows a cut perspective partial representation of a coil arrangement with plastic properties. The electrically conductive coil winding 66 can be seen within the coil arrangement 36. In addition, two strands 68 of a plastic material are provided in parallel to the coil winding 66, which renders the entire coil arrangement 36 plastically flexible.

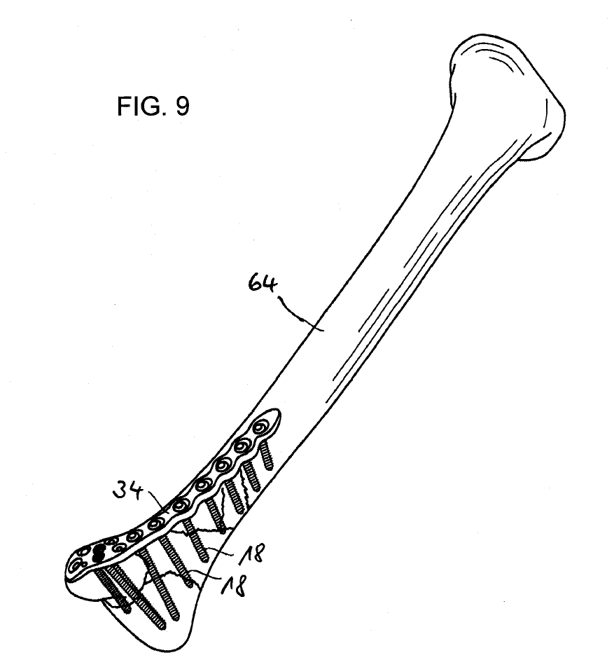

FIG. 9 shows an arrangement comprising a plurality of implant components for explaining another aspect of the invention. In osteosyntheses of, for example, a comminuted fracture by means of a plurality of screws, each of which is provided with a pick-up coil, it is possible that the pick-up coils are at different directions relative to the direction of the inducing magnetic field, and therefore, different electric voltages are activated at the screws. This is illustrated In FIG. 9 by the example with a bone support plate 34 fixed on the bone 64 by means of screws 18. The control in terms of an increase of the induced electric voltage (U_{ind}=dB/dt) should, therefore, be continued until all pick-up coils of the bone screws have—irrespective of their direction relative to the direction of the inducing EMF—reached the target voltage of 700 mV. As a protection from an undesired induction of more than 700 mV, the output of each pick-up coil is bridged by a diode having a block voltage of 700 mV.

The features of the invention disclosed in the above description, in the drawings as well as in the claims may be important for the realisation of the invention individually as well as in any combination.

### LIST OF NUMERALS

10 affected body region

12 control unit

14 input interface/reading device

16 bone screw/implant

18 screw head

20 thread

22 insulating region

24 transformer coil/transformer coil arrangement

26 magnetically soft core

28 sensing device

30 leg

32 RFID transponder/transmission device/transmitter

34 bone plate

36 coil arrangement/magnetic coil/coil

38 intersection point

40 surface

42 surface

44 magnetic field/magnetic field vector/vector symbol

46 magnetic field/magnetic field vector/vector symbol

60 coupling device

62 functional power generator

64 bone

66 coil winding

68 plastic strand

1. Apparatus for stimulating a healing process comprising:
a coil arrangement coupled to a functional power generator for generating an electromagnetic field in an affected body region;
a control unit for influencing a voltage curve generated by the functional power generator dependent on signals transmitted to an input interface of the control unit;
at least one implant positioned in the affected body region and coupled to a transformer coil arrangement, the poles of the transformer coil arrangement being connected to a pair of electrodes;
a sensing device for detecting an electric voltage existing between the electrodes of the pair of electrodes; and
a transmission device for transmitting signals characteristic to the electric voltage to the control unit.

2. Apparatus according to claim 1, wherein the transmission device comprises at least one transmitter for a wireless communication with a receiver associated with the input interface of the control unit.

3. Apparatus according to claim 1 wherein the transmission device comprises at least one RFID transponder the information content of which is detectable by a reading device associated with the input interface of the control unit.

4. Apparatus according to claim 3, wherein the readable information content of the RFID transponder is changeable depending on signals supplied by the sensing device.

5. Apparatus according to claim 3 wherein a plurality of RFID transponders is provided which can be activated or deactivated depending on signals supplied by the sensing device.

6. Apparatus according to claim 1, wherein the coil arrangement comprises a coil comprising a coil winding having an intersection point which defines two surfaces of a figure eight shaped form, the surfaces being aligned with respect to each other so that the magnetic fields generated by a current flow in the coil arrangement and pervading the surfaces are substantially rectified.

7. Apparatus according to claim 6, wherein the coil arrangement is flexible so that the surfaces are positionable at opposite sides of the affected body region.

8. Apparatus according to claim 1, wherein the functional power generator is capable of generating virtually purely harmonic voltage curves having a first harmonic wave component or abnormally harmonic voltage curves having a second harmonic wave component which is greater than the first harmonic wave component depending on signals transmitted to the input interface of the control unit.

9. Apparatus according to claim 8, wherein the functional power generator is capable of generating virtually purely harmonic voltage curves in the presence of a voltage detected by the sensing device which corresponds to a target voltage or is within a target voltage interval, and of generating abnormally harmonic voltage curves when the voltage falls below the target voltage or the target voltage interval.

10. Apparatus according to claim 9, wherein the functional power generator is integrated in a control that regulates the voltage detected by the sensing device to a target voltage or a target voltage interval by changing the voltage curves between purely harmonic and abnormally harmonic.

11. Apparatus according to claim 1, wherein a plurality of implants, corresponding transformer coil arrangements, corresponding pairs of electrodes, corresponding sensing devices and corresponding transmission devices are provided, and further wherein the functional power generator is influenceable on the basis of the lowest voltage measured by one of the sensing devices or the lowest voltage measured by one of the sensing devices which is still higher than a minimum voltage value, and further wherein voltage limiters are allocated to the pairs of electrodes.

12. Apparatus according to claim 3, wherein the transmission device comprises at least one RFID transponder the information content of which is detectable by a reading device associated with the input interface of the control unit.

13. Apparatus according to claim 4, wherein a plurality of RFID transponders is provided which can be activated or deactivated depending on signals supplied by the sensing device.
